(19) United States
(12) Patent Application Publication
(10) Pub. No.: US 2019/0353649 A1
(43) Pub. Date: Nov. 21, 2019

(54) POLYMER CONJUGATES, METHODS OF MAKING POLYMER CONJUGATES, AND METHODS OF USING POLYMER CONJUGATES

(71) Applicant: University of Florida Research Foundation, Inc., Gainesville, FL (US)

(72) Inventors: ADAM G. MONSALVE, Gainesville, GA (US); JON P. DOBSON, GAINESVILLE, FL (US)

(21) Appl. No.: 16/461,612

(22) PCT Filed: Dec. 1, 2017
(86) PCT No.: PCT/US2017/064112

§ 371 Date: May 16, 2019

Related U.S. Application Data
(60) Provisional application No. 62/428,575, filed on Dec. 1, 2016.

(51) Int. Cl. G01N 33/543; H01F 1/00; H01F 1/03

(52) U.S. Cl. CPC G01N 33/5434; H01F 1/0054; H01F 1/0315; C08F 292/00; G01N 2446/20; G01N 2446/84; H01F 1/0317

(57) ABSTRACT

Embodiments of the present disclosure provide for polymer conjugates, methods of making the polymer conjugates, methods of using polymer conjugates, and the like, where the polymer conjugates include magnetic particles (e.g. iron oxide particles). Embodiments of the present disclosure can be advantageous for one or more of the following reasons: strong and rapid magnetic response, multiple types of agents can be attached to the polymer conjugate, the size of the polymer conjugate can be controlled, and the polymer conjugates can be produced in a cost-effective manner.

### CLAIM OF PRIORITY TO RELATED APPLICATION

This application claims priority to co-pending U.S. provisional application entitled “POLYMER CONJUGATES, METHODS OF MAKING POLYMER CONJUGATES, AND METHODS OF USING POLYMER CONJUGATES” having Ser. No. 62/428,575, filed on Dec. 1, 2016, which is entirely incorporated herein by reference.

### GOVERNMENT FUNDING

This invention was made with government support under R21 EB020807 awarded by the National Institutes of Health. The government has certain rights in the invention.

### BACKGROUND

Magnetic micro- and nano-particle-based technologies can be used as a magnetic particle platform to collect biological entities. For example, the magnetic particle platform can include ligands selected based on their ability to specifically target bio-molecules of interest in a mixture. Subsequently, the magnetic particle platform can be separated from the mixture and studied.

### SUMMARY

Embodiments of the present disclosure provide for polymer conjugates, methods of making the polymer conjugates, methods of using polymer conjugates, and the like, where the polymer conjugates include magnetic particles (e.g. iron oxide particles).

Aspects of the present disclosure include a composition, among others, that includes: a polymer conjugate having a plurality of magnetic nanoparticles dispersed in a polymer microparticle, wherein the magnetic nanoparticles optionally have a coating, wherein the coating has the characteristic of providing stability for the magnetic nanoparticle in an aqueous solution. In an aspect, the polymeric microparticle is made of a polymer selected from the group consisting of: PLA, PGA, PLGA, PCL, poly(trimethylene carbonate) (PTMC), and a combination thereof. In an aspect, the magnetic particle is a material represented by M^{a}ₓM^{b}₍₁₋ₓ₎Fe₂O₄, where M is Fe, Co, Mn, Zn, Ta, Sr, or Ni, wherein x is 0 to 1. In an aspect, the coating is made from a material selected from the group consisting of: oleic acid, dimercaptosuccinic acid, citric acid, and a combination thereof.

An aspect of the present disclosure provides for a method of making a polymer conjugate, among others, that includes: providing a solution of magnetic nanoparticles, wherein the magnetic nanoparticle has a coating, wherein the coating has the characteristic of providing stability for the magnetic nanoparticle in an aqueous solution; mixing the solution of magnetic nanoparticles with a solvent having a polymer dissolved in the solvent to form a homogeneous solution; mixing the homogeneous solution with a solution of water; and forming the polymer conjugates. In addition, an aspect includes a composition comprising a polymer conjugate of the method descried above.

An aspect of the present disclosure provides for a method of separation, comprising: exposing a polymer conjugate to a mixture, wherein the polymer conjugate having a plurality of magnetic nanoparticles dispersed in a polymer microparticle, wherein the magnetic nanoparticles optionally have a coating, wherein the coating has the characteristic of providing stability for the magnetic nanoparticle in an aqueous solution, and wherein an agent attached to the surface of the polymer conjugate, wherein the agent has an affinity for a target, wherein the mixture optionally comprises the target; bonding the target to the agent of the polymer conjugate; and separating, magnetically, the polymer conjugate from the mixture.

### BRIEF DESCRIPTION OF THE DRAWINGS

Many aspects of the present disclosure can be better understood with reference to the following drawings.

FIG. 1 illustrates a depiction of three molecules grafted to the PLA/magnetic composite particle surface.

FIG. 2 illustrates the magnetic microparticle characterization through a) light microscopy, b) dynamic light scattering, and c) SQUID magnetometry.

FIG. 3 illustrates flow cytometric analysis of his-tagged GFP magnetic capture following incubation with PLA particles with and without the Nickel NTA surface functionalization.

FIG. 4 illustrates the confirmation of FITC-BSA functionalization with flow cytometry (left) comparing bare (red) and FITC BSA (blue) and fluorescence microscopy (right).

FIG. 5 illustrates ELISA analysis of TNF alpha capture comparing Anti-TNF-alpha conjugated PLA microspheres with bare PLA microspheres.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method may be carried out in the order of events recited or in any other order that is logically possible.

Embodiments of the present disclosure will employ, unless otherwise indicated, techniques of chemistry, biochemistry, polymer chemistry, and the like, which are within the skill of the art. Such techniques are explained fully in the literature.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Discussion

Embodiments of the present disclosure provide for polymer conjugates, methods of making the polymer conjugates, methods of using polymer conjugates, and the like, where the polymer conjugates include magnetic particles (e.g. iron oxide particles). Embodiments of the present disclosure can be advantageous for one or more of the following reasons: strong and rapid magnetic response, multiple types of agents can be attached to the polymer conjugate, the size of the polymer conjugate can be controlled, and the polymer conjugates can be produced in a cost-effective manner. In an aspect, the polymer conjugates can be used biomedical applications such as biomacromolecule isolation, cell separation, heavy metal toxin removal, and as a fluorescent imaging modality.

Embodiments of the present disclosure provide for the ability to apply magnetic fields onto the magnetic particles (e.g., in a polymer conjugate) to separate the polymer conjugate (and a target attached thereto) from the remaining sample (e.g., mixture). In an embodiment, the magnetic field can be generated by electrically conducting coils connected to a power source, or by high-gradient permanent magnets or magnetic materials such as NdFeB, AlNiCo, or SmCo magnets.

Embodiments of the present disclosure can include a polymer conjugate having a plurality of magnetic nanoparticles dispersed in a polymer microparticle. In an aspect, the magnetic nanoparticle can have a coating, where the coating has the characteristic of providing stability for the magnetic nanoparticle in an organic or an aqueous solution, depending on the selected coating, to produce the polymer microparticle. In another aspect, the magnetic nanoparticle does not have a coating. In an aspect, the polymer microparticle is made of a polymer and the magnetic nanoparticles are disposed within the polymer microparticle, while a portion of the magnetic nanoparticles can optionally have an area on the surface of the polymer microparticle that is not enclosed by the polymer. In an embodiment, an agent can be attached to the surface of the polymer conjugate, where the agent has an affinity for the target (e.g., biomolecule, heavy metal toxin, and the like) of interest.

In an embodiment, the polymer microparticle can have a diameter of about 0.1 μm to 100 μm. In an embodiment, the polymer conjugate can include about 1% loading to 80% loading of the magnetic nanoparticles. In an embodiment, the polymer conjugate having agents attached thereto can have a diameter of about 0.1 to 150 μm.

In an embodiment, the polymer microparticle can be made of a polymer or block copolymers of: poly(ethylene glycol), poly(oxazolines), poly(ε-caprolactone) (PCL), poly(D/L-lactic acid) (PLA), poly(glycolic acid) (PGA), poly(lactic-co-glycolic acid) (PLGA), acrylamide-based monomers, methacrylamide-based monomers, acrylate-based monomers, methacrylate-based monomers, including, for example, N-(2-hydroxypropyl) methacrylamide and maleimide functional methacrylate, poly (β-thioesters), or random copolymers of hexyl methacrylate and maleimide functional methacrylate. In an embodiment, the polymer microparticle can be made of polymers such as: PLA, PGA, PLGA, PCL, poly(trimethylene carbonate) (PTMC), and a combination thereof. In an embodiment, the polymer can have a molecular weight of about 1,000 g/mol to 100,000 g/mol.

In an embodiment, the magnetic particle has a magnetic moment strong enough to accomplish the desired result (e.g., separation). In an embodiment, the magnetic particle can include iron, cobalt, nickel, oxides of each, or combinations thereof. In an embodiment the magnetic particle can be represented by M^{a}ₓM^{b}₍₁₋ₓ₎Fe₂O₄, where M is Fe, Co, Mn, Zn, Ta, Sr, or Ni, wherein x is 0 to 1. In an embodiment, the magnetic particle can be: iron oxide, Fe₃O₄, γFe₂O₃, αFe₂O₃. In an embodiment, the magnetic particle can be SrFe₁₂O₁₉ or BaFe₁₂O₁₉. In an embodiment, the magnetic particle can have a diameter on the nano-scale of about 5 to 500 nm, about 10 to 200 nm, about 10 to 100 nm, or about 10 to 50 nm.

In an embodiment, the magnetic nanoparticle can have a coating on a portion (e.g., about 20 to 95% of the surface) of the nanoparticle or covering the entire nanoparticle. In an embodiment, the coating can be made of a material such as: oleic acid, polyethylene glycol (PEG), dimercaptosuccinic acid (DMSA), citric acid, and a combination thereof. In an embodiment, the coating can have a thickness of about 0.1 nm to 30 nm.

As mentioned above, the polymer conjugate can include one or more agents (e.g., a chemical or biological agent) bonded (e.g., directly or indirectly via a ligand) to thereto. The term “bind”, “bond”, or “bound” can refer to, but is not limited to, chemically bonded (e.g., covalently or ionically), biologically bonded, adsorbed via charge interactions, biochemically bonded, and/or otherwise associated with the material. In an embodiment, “bonded” can include, but is not limited to, a covalent bond, a non-covalent bond, an ionic bond, a chelated bond, as well as being bound through interactions such as, but not limited to, hydrophobic interactions, hydrophilic interactions, charge-charge interactions, π-π stacking interactions, combinations thereof, and like interactions. In an embodiment the polymer conjugate can include a linker (e.g., a hydrocarbon chain, polymer, and the like) and/or coating (e.g., a polymer or the like) so that the agent can bind to the polymer conjugate.

In an aspect, the agent can be used to treat, image, detect, study, monitor, and/or evaluate a condition or an occurrence, or the like in the subject. In an embodiment, the agent can include, but is not limited to, a drug, a therapeutic agent, a radiological agent, a fluorescent agent, a small molecule drug, a biological agent (e.g., polypeptides (e.g., proteins such as, but not limited to, antibodies (monoclonal or polyclonal)), antigens, nucleic acids (both monomeric and oligomeric), polysaccharides, haptens, sugars, fatty acids, steroids, purines, pyrimidines, ligands, and aptamers) and combinations thereof, that can be used to image, detect, study, monitor, evaluate, and the like. In an embodiment, the agent is included in an effective amount to accomplish its purpose, where such factors to accomplish the purpose are well known in the medical arts.

In an embodiment, the polymer conjugate can include an agent that is a targeting agent, where the targeting agent has an affinity for a target (e.g. a target cell, tissue, tumor, or biological component associated with any of these). “Affinity” as used herein refers to the targeting agent having a stronger attraction towards the target (e.g., biomolecule, cell, and the like) relative to other components of the mixture. In an embodiment, the targeting agent can include, but is not limited to, a chemical agent, a biological agent (e.g., polypeptides (e.g., proteins such as, but not limited to, antibodies (monoclonal or polyclonal)), antigens, nucleic acids (both monomeric and oligomeric), polysaccharides, haptens, sugars, fatty acids, steroids, purines, pyrimidines, ligands, and aptamers) and combinations thereof, that have an affinity for the target. In an embodiment, the agent can be a metal chelate such as nitro-triacetic acid, capable of coordinating nickel ions, which display a specific affinity towards proteins containing polyhistidine residues.

In an embodiment, the polymer conjugate can be made using the following method. A solution of magnetic nanoparticles (e.g., iron oxide nanoparticles) is provided, where the magnetic particle has a coating (such as described herein). The solution of magnetic nanoparticles is mixed (e.g., for about 10 to 30 min) with a solvent having a polymer dissolved in the solvent to form a homogeneous solution. In an embodiment, the solvent is an organic solvent such as dichloromethane, chloroform, or a combination thereof. In an embodiment, the concentration of the polymer in the solvent can be about 10 mg/mL to 100 mg/mL. Subsequently, the homogeneous solution can be mixed via an ultrasonic homogenizing tip (e.g., for about 30 seconds to 3 min) with a solution of water to form the polymer conjugates. In an embodiment, the ratio of homogeneous solution to water can be about 4:1 to 10:1.

In an embodiment of the method, in the first emulsion a small volume of water is added to the magnetic particle/polymer solution. Following a period of sonication, the entire volume of liquid is decanted into a larger volume (e.g., about 10×) of polyvinyl alcohol, in water (5% solution). The second solution is then sonicated for a set period of time and the mixture is stirred on a magnetic stir plate overnight to allow for the organic solvents to fully evaporate.

After the polymer conjugate is formed one or more agents can be attached (e.g., bonded directly or indirectly via a linking molecule) to the surface of the polymer conjugate. In an aspect, the number of agents attached to the surface of the polymer conjugate can depend upon the surface area of the polymer conjugate and can generally vary from 2 to 1,000.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### Example 1

Iron oxide nanoparticles have been employed for a variety of biomedical applications in both the diagnostic and therapeutic realm and the number of applications continues to grow. In this example, magnetic nanoparticles were synthesized via aqueous co-precipitation and subsequently coated in oleic acid to confer colloidal stability in organic solvents. The synthesized nanoparticles were encapsulated within poly(lactic acid) via a double emulsion synthesis. Following the microparticle synthesis, surface functionalization experiments were performed to gain an understanding of the surface chemistry and the potential ability to conjugate biomacromolecules to the particle surface for use in biomedical applications. The ability to specifically bind to histidine tagged proteins was assessed through flow cytometry analysis of histidine-tagged green fluorescent protein after incubation with the particles. The magnetic separation of pro-inflammatory proteins was assessed using enzyme-linked immunosorbent assays to monitor cytokine depletion from known concentrations of tumor necrosis factor-alpha. Lastly, a fluorescently tagged protein molecule was bound to the surface, which allowed the proteins to be observed visually under a fluorescent microscope. The particles described here have the potential for numerous biomedical applications including biomacromolecule isolation, cell separation, heavy metal toxin removal, and as a fluorescent imaging modality.

### Introduction

### A. Magnetic Particles

Magnetic nanoparticle-based technologies continue to grow in their usage in a number of biomedical applications such as MRI contrast enhancement, triggered drug delivery, and cancer therapy with magnetic fluid hyperthermia. [1-3] The focus of this paper, however, is restricted to the development of a magnetic particle platform to collect biological entities such as cells, nucleic acids, or proteins. [4-6] Applications of magnetic particles in biomedicine depend upon, or can be improved by, the conjugation of the particles with biological ligands such as antibodies, enzymes, aptamers or microRNA that allow for the specific targeting of a bio-molecular target. Once the desired target agent has been bound to the particle, the magnetic properties of the particle allow for separation out of a larger pool of biomolecules. This utility of magnetic particles has proven quite advantageous as molecular biologists have adopted this method to quickly isolate or purify biological molecules of interest.

In this example, microspheres composed of poly(lactic acid) PLA, were synthesized containing embedded iron oxide nanoparticles to confer the particles with magnetic properties. PLA was chosen due to its biodegradability, slow degradation kinetics, surface chemistry amenable to ligand functionalization, and the fact that biomaterials composed of PLA have been approved by the U.S. Food and Drug Administration (FDA). [7-9] The iron oxide nanoparticles were synthesized using an aqueous co-precipitation adapted from Mandavi et. al yielding magnetic nanoparticles with colloidal stability in organic solvents. [10] The microparticles were synthesized using either a single or double emulsion followed by solvent evaporation.

For the microparticle synthesis via emulsion-solvent evaporation, a sonicator tip delivers energy to form an emulsion between an aqueous phase and an organic phase that induces the formation of the microparticles. The technique requires a surfactant molecule to allow for the formed particles to be stable in solution and not aggregate during the synthesis process. The properties of the synthesized particles can be tuned based upon a number of synthetic conditions including the sonication intensity and time, concentration of surfactant, polymer molecular weight, and volumes of each phase. For this synthetic process, the surfactant chosen to stabilize the emulsion was poly(vinyl alcohol). The synthesis method has the advantages of being simple, easy to reproduce, scalable, and does not require expensive equipment.

### B. Immobilized Metal Ions for Tagged Protein Isolation

The ability to produce and isolate a spectrum of proteins, ranging from antibodies to signaling proteins, has become essential in the field of molecular biology. In metal affinity chromatography, a tag or residue is added to the targeted protein and then the sample solution is run through a column containing the binding partner, usually an immobilized transition metal such as nickel. The result is the formation of coordination compounds where ligands coordinate around a central metal ion. These coordination compounds can therefore specifically isolate the protein of choice out of a larger pool of biomolecules. In particular, coordination complexes of nickel ions have seen significant utilization in recombinant protein technology through the usage of polyhistidine tags. [12-14] The imidazole ring has electron donor groups that readily form coordination bonds with immobilized transition metal ions. Histidine exhibits a strong affinity for a number of transition metals ions including cobalt, copper, and zinc with the highest affinity for nickel ions. With advances in molecular biology allowing for the fairly easy modification of proteins, the addition of the amino acid histidine is now relatively straightforward.

The current use of chelated nickel to bind histidine tagged proteins has generally been limited to applications of protein purification in molecular biology. However, the fact that the proteins can be easily removed and quantified following capture using an imidazole wash makes this bonding mechanism a useful one for in vitro experiments. For the current work, the goal was to develop particles that could be loaded at their surface with partially coordinated nickel ions that allowed for the subsequent binding of histidine-tagged protein molecules for potential applications of cytokine removal. Another useful application for microparticles that can chelate metal ions may be in heavy metal ion removal from contaminated water sources to remove Pb or additional toxic heavy metal ions. PLA contains carboxyl groups that make it amenable to surface functionalization with proteins or additional ligands and small molecules, making this microparticle dynamic in the range of potential targets and uses.

### C. Cytokine Collection and Fluorescence Imaging

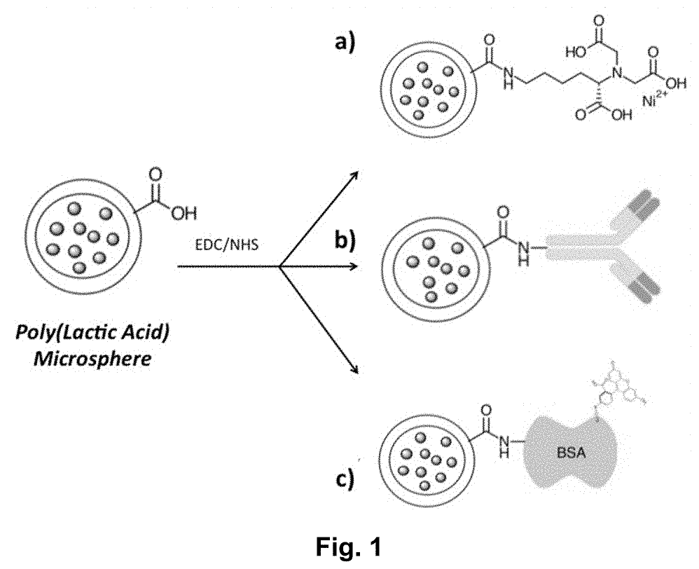

In addition to the scavenging of histidine tagged proteins, magnetic particles can be used to collect non-tagged proteins such as growth factors, cytokines, and chemokines. For untagged protein collection, the binding requires the targeting agent have a natural affinity towards the target molecule. In general the type of molecules used for this purpose are antibodies, which can be engineered to target specific targets. Newer molecules such as nucleic acid aptamers have begun to be utilized as targeting molecules.[16] For the binding of these molecules to the particle surface, the antibody can be cross-linked through amine, carboxylic acid, or thiol functional groups found in protein molecules. When crosslinking proteins to a surface, however, protein function must be assessed following the binding as the crosslinking may change the structure of the protein in a way that alters its function. The carboxylic acid functional group present on the surface of PLA microparticles can easily be linked to amine, carboxyl, or thiol functional groups that are present on a number of amino acid side chains as well as the C and N terminus. For the current work, EDC/NHS crosslinking chemistry covalently attached Nickel-nitro-triacetic Acid (Ni-NTA), fluorescein-isothiocyanate-bovine serum albumin (FITC-BSA), and Anti-Tumor Necrosis Factor-alpha (AntiTNF-α) molecules to the surface as depicted in FIG. 1. The binding of each of these ligands was assessed through flow cytometry, fluorescence microscopy and enzyme linked immune-sorbent assay (ELISA).

### II. Materials and Methods

Iron(II) chloride tetrahydrate, iron(III) chloride hexahydrate, poly(lactic acid), 2-(N-morpholino) ethanesulfonic acid (MES), oleic acid, 1-ethyl-3-(3-dimethylaminopropyl) carbodiimide (EDC), N-hydroxysulfosuccinimide (Sulfo-NHS), chloroform, dichloromethane, phosphate buffered saline (PBS), poly(vinylalcohol) (M_{w} 50,000 g/mol), poly(lactic acid) (M_{w} 60,000 g/mol), fluorescein-bovine serum albumin, N,N-bis(carboxymethyl)-L-lysine hydrate, and nickel chloride were purchased from Sigma Aldrich. Recombinant Aequorea victoria his-tagged green fluorescent protein (his-GFP) was purchased from Thermo Fisher Scientific. The ELISA kit for hTNF α as well as the hTNFα cytokine were purchased from RnD Sciences. Imaging was performed on an EVOS FL light microscope. Dynamic light scattering (DLS) measurements made on Brookhaven ZETAPALS. Transmission electron micrographs were taken with a Hitachi 7600. X-Ray Diffraction was performed on a Panalytical XPert diffractometer. Thermogravimetric Analysis was performed on a TA Instruments SDTQ600. Flow cytometric analysis was performed on a Sony iCyte Eclipse and analyzed with FlowJo. Particles were lyophilized in an Labconco Free Zone 4.5 freeze drier. Magnetic measurements were made using a Quantum Design MPMS-XL7 magnetometer based on a superconducting quantum interference device (SQUID). Spectrophotometric analysis for the UV-Vis Iron quantification was performed on a Biotek Synergy 2 spectrophotometer

### A. Synthesis of Iron Oxide Nanoparticles

The magnetic nanoparticles were synthesized using a simple co-precipitation method adapted from Mandavi et al. [1] Briefly, 3.36 g of iron(II)chloride tetrahydrate and 9.67 g of iron(III) chloride hexahydrate were dissolved in 250 mL distilled water. The solution was heated to 45° C. for 30 minutes with constant stirring. Subsequently, a solution of ammonium hydroxide (25%) was quickly added to the iron salts until a pH of 12 was reached upon which the solution turned to a deep black color. After this addition, 3 mL of oleic acid was added to the stirring solution and the solution temperature was immediately increased to 80° C. The solution was mixed at 80° C. for 1 hour. After the hour of heating, the solution was allowed to slowly cool to room temperature with continuous stirring. The particles were magnetically separated with a NdFeB magnet and the non-magnetic components of the solution were decanted. The particles were washed five times using water and ethanol as the wash solvents and were suspended in chloroform. The synthesized nanoparticles were characterized for their size and shape through transmission electron microscopy. The magnetic properties of the particles were characterized through SQUID magnetometry as well as colorimetric methods using o-phenanthroline. To confirm the inverse spinel structure of magnetite and maghemite, X-Ray diffraction was performed.

### B. PLA Microparticle Synthesis and Characterization

The first synthesis, a single emulsion-solvent evaporation, began by dissolving the 500 mg of PLA in 10 mL of dichloromethane for 30 minutes to allow for the complete dissolution of the polymer. While the polymer was dissolving, 150 mL of a 5% PVA solution in double distilled water (ddH₂O) was prepared in a beaker containing a magnetic stir bar. 2 mL of the agnetic nanoparticles (50 mg/mL) suspended in chloroform were added to the polymer solution once dissolved and allowed to mix for 10 minutes to ensure a homogenous suspension. Following this, the entire volume of the organic phase was added to the 150 mL PVA solution. Immediately the solution was subjected to sonication for 60 seconds to form the emulsion. The solution was placed on a stir plate and allowed to stir overnight using a magnetic stir bar at 600 RPM. The following day, the particles were centrifuged at 1000 RPM to remove the large particles and the magnetic PLA microparticles were isolated using a NdFeB array. The particles were suspended in 5 mL of water and lyophilized to yield a dry sample.

For the double emulsion, prior to the microparticle synthesis, 500 mg of PLA was dissolved into 10 mL of dichloromethane by placing the beaker on a shake plate and stirring for 30 minutes. The beaker was covered to keep the volatile organic solvent from escaping. 2 mL of the 50 mg/mL iron oxide solution was then added to the PLA/chloroform solution and mixed thoroughly to form a homogenous solution of magnetic particles and polymer. For the first emulsion, 1.5 mL of water was added to the prepared organic phase, containing iron oxide and PLA, and immediately sonicated for 30 seconds This emulsified solution was then added to 150 mL of 5% aqueous PVA and sonicated a second time for 30 seconds. The prepared solution was then stirred at 600 RPM overnight to allow the organic solvents to evaporate. The following day, the particles were centrifuged (10 mins, 1000 RPM) to remove the larger particles and the supernatant was placed onto a NdFeB magnet array in order to isolate the magnetic microparticles. The isolated magnetic particles were then suspended in distilled water and lyophilized to yield a dry sample.

Following synthesis, the microparticles were characterized to determine the available functional groups, size distribution and magnetic properties. FTIR was used to confirm the presence of the PLA matrix as well as the presence of a carboxylic acid functional group that would be crucial to conjugating biologically functional molecules to the particle surface. Size distribution of the particles was examined with ImageJ analysis of light microscope images as well as dynamic light scattering measurements.

C. Conjugation with Ni-NTA, Anti-TNF-α, and FITC-BSA

For coupling reactions, the PLA microparticles were magnetically washed three times with MES buffer, pH 4.7, to pre-equilibrate them in the proper buffer at the appropriate pH for conjugation. Once equilibrated, the particles were subjected to EDC and Sulfo NHS dissolved in MES buffer for 15 minutes with constant agitation at room temperature. Following the EDC/NHS activation, the microparticles were magnetically separated and then incubated in the N,N-Bis(carboxymethyl)-L-lysine (to capture his-tagged GFP), TNF-α antibody (to capture TNF-α), or FITC-BSA (to demonstrate BSA functionalization) in PBS, pH 7.2, overnight at 4° C. under constant gentle mixing. Following the overnight crosslinking, the particles were separated from any unbound molecules via magnetic separation and resuspended two times with tris-buffered saline prior to a 15-minute incubation in tris-buffered saline to quench any unreacted carboxylic acids. Following this incubation, the particles were magnetically washed 3 times and suspended in PBS. For the Nickel-NTA conjugation, the particles were subsequently loaded with Nickel Chloride

D. Flow Cytometry Analysis of his-GFP and FITC-BSA

Following characterization, experiments to determine the viability of these particles are scavengers of his-tagged proteins were performed. His-GFP containing an N terminal histidine tag was utilized to verify the binding of the protein to the particles. The use of his-GFP allows for particles that have bound the proteins to be quantified via flow cytometry analysis of the fluorescence intensity. The experimental setup consisted of both Ni-NTA conjugated and unconjugated PLA microparticles as a control. The particles were incubated with the his-GFP or blank buffers for 30 minutes with constant gentle agitation. Following the incubation period, the particles were magnetically washed with PBS two times prior to being suspended in PBS+1% BSA for flow cytometric analysis. The particles were gated using a control sample of non-functionalized particles to determine the location on the scatter plot containing the particles and this gate was used for the additional samples. Data analysis was performed on FlowJo.

In addition, flow cytometry analysis was performed to assess the conjugation of FITC-BSA to the particle surface. Both FITC-BSA conjugated particles and unconjugated particles were washed three times with PBS and resuspended in PBS. The particles were assessed for fluorescence intensity and compared with control particles not conjugated with the FITC-BSA molecules.

### E. Evaluation of TNF-α Scavenging

To test the ability to capture TNF-α, a TNF-α solution was created with a known concentration of 100 pg/mL. The conjugated magnetic nanoparticles were then incubated in the solution of the known concentration of TNF-α. The concentration of particles were used for the depletion experiments was 60 μg/mL and once the particles were mixed with the cytokines, the solution was gently mixed on a shaker plate. Following an incubation period, the particles were then magnetically separated using a NdFeB permanent magnet for 10 minutes. The supernatant was removed and saved for use in the ELISA to quantify the TNF-α levels.

### III. Results and Discussion

### A. Iron Oxide Nanoparticles

Magnetic nanoparticles were synthesized and successful coordination of the oleic acid onto the iron oxide surface was evident due to the stability of the iron oxide nanoparticles in organic solvents such as chloroform and dichloromethane. The particles exhibit significant long-term colloidal stability in the organic solvents for over 12 months. The crystal structure of the synthesized nanoparticles was confirmed to match the inverse spinel spectrum characteristic of magnetite and maghemite crystals. Transmission electron microscopy showed that the nanoparticles exhibited polydispersity with an average particle diameter between 8-15 nm. SQUID magnetometry yielded a saturation magnetization of 84.72 emu/g for the oleic acid coated nanoparticles.

### B. Magnetic Microparticles

Analysis of microparticles revealed the average particle diameter from a total number of 600 particles was about 4.8 μm. Particles subjected to centrifugation at 2000 RPM instead of 1000 RPM exhibited slightly smaller average diameter around 3.5 μm. The particles were magnetically separated within one minute of magnetic field exposure, indicating significant levels of magnetic particle encapsulation.

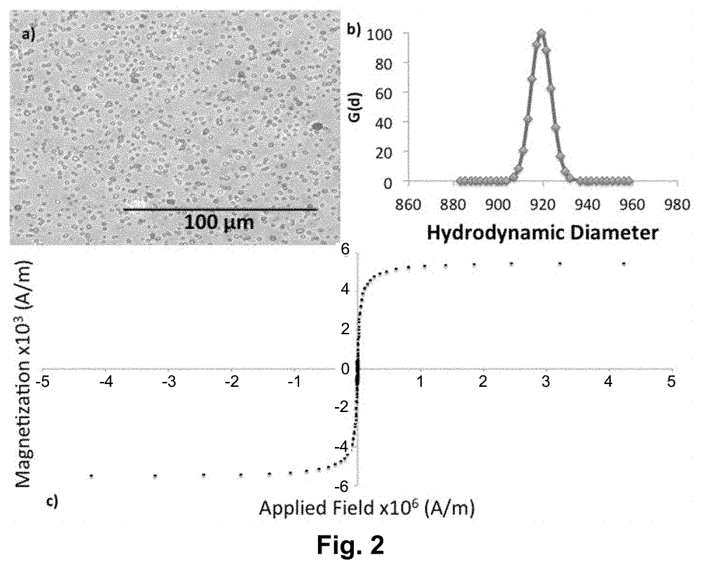

In order to quantify the amount or iron oxide loading we performed thermo-gravimetric and Superconducting Quantum Interference Device (SQUID) analysis. Thermo gravimetric analysis showed that after heating the sample to 800° C., where all components except iron oxide should be vaporized, more than 5% of the initial sample weight remained. Magnetization vs. field curves showed a saturation magnetization of −5.5 emu/g as shown in FIG. 2. Studies of pure iron oxide nanoparticles have shown a saturation point of around 70 emu/g. [17] Using this saturation value would indicate that the microparticles are composed of approximately 8% iron oxide by weight.

### D. Ni-NTA and FITC-BSA Functionalization

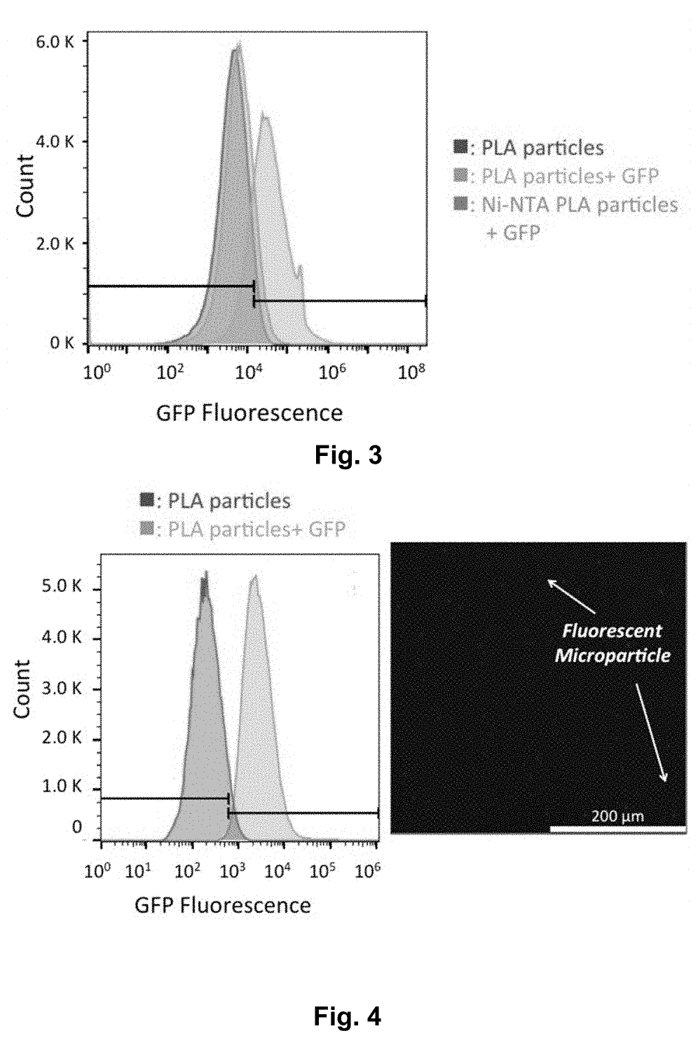

Confirmation of Ni-NTA functionalization is shown in FIG. 3. A significant increase from approximately 6×10³ to 2×10⁴ fluorescence units is observed in the Ni-NTA surface functionalized particles compared with bare PLA microparticles. The fact that the unconjugated particles did not show significant increases after incubation with GFP compared with the conjugated particles helps to illustrate that the particles are specific for his-tagged proteins and will not non-specifically bind other proteins.

Flow cytometric confirmation of FITC-BSA surface conjugation is shown in FIG. 4. The sample functionalized with FITC-BSA molecules exhibited significantly higher fluorescence intensity at about 3×10³ compared with unconjugated particles with fluorescence intensity of 2×10².

### E. Evaluation of TNF-α Scavenging

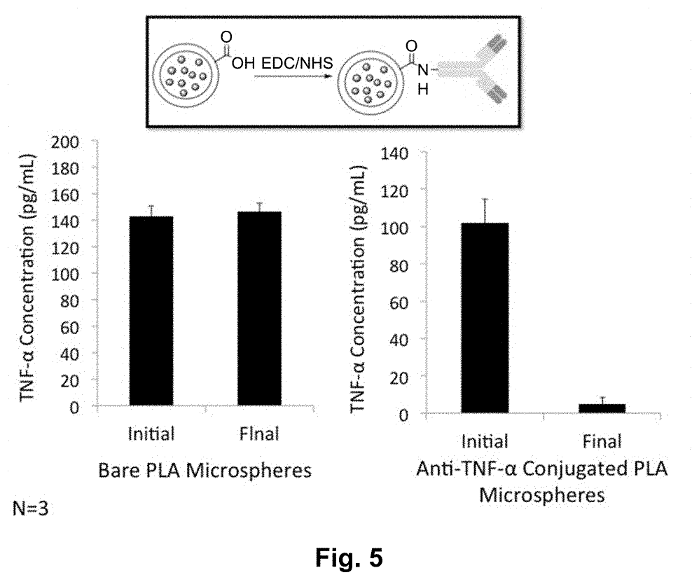

Experiments testing the magnetic separation of TNF-α show clearly that the particles bind and remove TNF-α from solution. The particles (60 μg/mL) were incubated with the initial solution containing known levels of TNF-α for 12 hours to allow for complete binding of the cytokine. The results shown in FIG. 5 indicate virtually zero removal when using particles with no surface modifications while the 33 and 66 ug/mL concentrations of functionalized particles achieved upwards of 70% and 80% collection, respectively.

### IV. Conclusion

Composite iron oxide-PLA microparticles were synthesized and characterized. Our method proved to effectively produce colloidally stable magnetic nanoparticles reducing the cost associated with many of the commercially available iron oxide nanoparticle formulations. The magnetic loading, crystal structure, and average particle size were characterized along with other physical properties. The particles were subsequently functionalized with three different ligand molecules, Ni-NTA, FITC-BSA, and Anti-TNF-α. Capture of his-GFP was verified through flow cytometry analysis comparing unconjugated particles with particles functionalized with NTA and loaded with nickel. The FITC-BSA conjugation was also verified with flow cytometry as well as fluorescent microscopy. Lastly, antibody functionalization was confirmed using an ELISA to quantify the removal of TNF-α from a known concentration of the cytokine. The ability to readily functionalize the surface of the microparticles helps to illustrate the variety of potential biomedical applications.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

We claim:

1. A composition, comprising: a polymer conjugate having a plurality of magnetic nanoparticles dispersed in a polymer microparticle, wherein the magnetic nanoparticles have a coating, wherein the coating has the characteristic of providing stability for the magnetic nanoparticle in an aqueous solution.

2. The composition of claim 1, wherein the polymeric microparticle is made of a polymer selected from the group consisting of: PLA, PGA, PLGA, PCL, poly(trimethylene carbonate) (PTMC), and a combination thereof.

3. (canceled)

4. The composition of claim 1, wherein the magnetic particle is a material represented by M^{a}ₓM^{b}₍₁₋ₓ₎Fe₂O₄, where M is Fe, Co, Mn, Zn, Ta, Sr, or Ni, wherein x is 0 to 1, optionally wherein the magnetic particle is a material selected from the group consisting of: Fe₃O₄, γFe₂O₃, αFe₂O₃, MnFe₂O₄, ZnFe₂O₄, and Mn_{0.5}Zn_{0.5}Fe₂O₄.

5. (canceled)

6. The composition of claim 1, wherein the magnetic particles have a diameter of about 5 to 500 nm.

7. The composition of claim 1, wherein the coating is made from a material selected from the group consisting of: oleic acid, dimercaptosuccinic acid, citric acid, and a combination thereof.

8. (canceled)

9. The composition of claim 1, further comprising an agent attached to the surface of the polymer conjugate, optionally wherein the agent includes a drug, a therapeutic agent, a fluorescent agent, a radiological agent, or a biological agent.

10. (canceled)

11. (canceled)

12. The composition of claim 1, wherein the polymer microparticle has a diameter of about 0.1 μm to 100 μm.

13. The composition of claim 1, wherein the polymer conjugate has about 1% loading to 80% loading of the magnetic nanoparticles.

14. (canceled)

15. A method of making a polymer conjugate, comprising:
providing a solution of magnetic nanoparticles, wherein the magnetic nanoparticle has a coating, wherein the coating has the characteristic of providing stability for the magnetic nanoparticle in an aqueous solution;
mixing the solution of magnetic nanoparticles with a solvent having a polymer dissolved in the solvent to form a homogeneous solution;
mixing the homogeneous solution with a solution of water; and
forming the polymer conjugates.

16. The method of claim 15, wherein the ratio of homogeneous solution to water is about 1:5 to 1:40.

17. The method of claim 15, wherein the solvent is an organic solvent, wherein optionally wherein the organic solvent is selected from the group consisting of: chloroform, dichloromethane, and a combination thereof.

18. (canceled)

19. The method of claim 15, wherein the polymeric microparticle is made of a polymer selected from the group consisting of: PLA, PGA, PLGA, PCL, poly(trimethylene carbonate) (PTMC), and a combination thereof.

20. (canceled)

21. The method of claim 15, wherein the magnetic particle is a material represented by M^{a}ₓM^{b}₍₁₋ₓ₎Fe₂O₄, where M is Fe, Co, Mn, Zn, Ta, Sr, or Ni, wherein x is 0 to 1.

22. The method of claim 21, wherein the magnetic particle is a material selected from the group consisting of: Fe₃O₄, γFe₂O₃, αFe₂O₃, MnFe₂O₄, ZnFe₂O₄, and Mn_{0.5}Zn_{0.5}Fe₂O₄.

23. The method of claim 22, wherein the magnetic particles have a diameter of about 5 to 500 nm.

24. The method of claim 15, wherein the coating is made from a material selected from the group consisting of: oleic acid, dimercaptosuccinic acid, citric acid, and a combination thereof.

25. The method of claim 24, wherein the coating comprises oleic acid.

26. The method of claim 15, wherein the polymer microparticle has a diameter of about 0.1 μm to 100 μm.

27. The method of claim 15, wherein the polymer conjugate has about 1% loading to 80% loading of the magnetic nanoparticles.

28. (canceled)

29. A method of separation, comprising:
exposing a polymer conjugate to a mixture, wherein the polymer conjugate having a plurality of magnetic nanoparticles dispersed in a polymer microparticle, wherein the magnetic nanoparticles have a coating, wherein the coating has the characteristic of providing stability for the magnetic nanoparticle in an aqueous solution, and wherein an agent attached to the surface of the polymer conjugate, wherein the agent has an affinity for a target, wherein the mixture optionally comprises the target;
bonding the target to the agent of the polymer conjugate; and
separating, magnetically, the polymer conjugate from the mixture.
